(19) United States
(12) Patent Application Publication
(10) Pub. No.: US 2022/0266261 A1
(43) Pub. Date: Aug. 25, 2022

(54) ELECTRIC FIELD PARTICLE SORTING DEVICE

(71) Applicant: XIDAS, INC., Irvine, CA (US)

(72) Inventors: Mark Bachman, Irvine, CA (US); Philip N Duncan, Irvine, CA (US); Sarkis Babikian, Irvine, CA (US)

(21) Appl. No.: 17/680,039

(22) Filed: Feb. 24, 2022

Related U.S. Application Data
(60) Provisional application No. 63/153,635, filed on Feb. 25, 2021.

(51) Int. Cl. B03C 5/00; C12N 13/00; B01L 3/00

(52) U.S. Cl. CPC B03C 5/005; C12N 13/00; B01L 3/502761; B01L 2200/0652; B01L 2300/0645

(57) ABSTRACT

The present invention describes a device for sorting small particles using electric fields. The device described herein comprises one or more electrically conducting structures suspended in a fluid flow stream used to redirect the movement of particles in the flow stream. The electrically conducting structures are longitudinally disposed at a center axis of a fluidic channel. As particles flow in the fluid, electric fields on the suspended conductors move the small particles from one flow region to another, allowing them to be redirected to different endpoints.

### CROSS-REFERENCES TO RELATED APPLICATIONS

This application is a non-provisional and claims benefit of U.S. Provisional Application No. 63/153,635 filed Feb. 25, 2021, the specification of which is incorporated herein in their entirety by reference.

### BACKGROUND OF THE INVENTION

Electric fields are often used for separating particles from a heterogeneous liquid media sample. Examples are electrophoresis and dielectrophoresis which separate particles based on charge or dipole moment. Electric field-flow fractionations (E-FFF) are continuous-flow processes that separate particles and cells within a flowing media based on their size and response to electric fields. Similar to stationary electrophoresis and dielectrophoresis, cells and particles migrate due to an externally applied field that preferentially moves a subpopulation within the sample. This migration is induced within a flowing media to push specific components along a desired trajectory for collection.

In a typical scenario, a heterogeneous sample is introduced at an inlet and mixed with a carrier media before flowing over a series of electrodes to outlets. Due to either slow flow velocity or small fluid channel size (or both), the flow regime within the system is laminar. Laminar flow streams do not mix, such that particles in the media follow predictable flow paths from inlets to outlets. Particles within the flow are carried along by the flow to one of the outlets. The effect is illustrated in FIG. 5.

As particles flow over or near the electrodes, an electrical signal is applied to the electrodes to create an electric field and electric field gradient, which in turn creates a force on some of the particles, as illustrated in FIG. 6. Each particle in the stream is subjected to the hydrodynamic forces from the movement of the fluid, the electric field, and gravity. As the effect from gravity is usually negligible, each particle's movement depends on its hydrodynamic, electric, and dipole forces. The greater the force effect of the electric field, the farther a specific particle will move. As a result, the particles in the flow will fractionate according to the properties of the individual particles. Small movements due to the electric fields capture and nudge the target particles to a different part of the stream so it is moved to a different outlet. The large flow drives target particles preferentially to this outlet, such that it has a higher proportion of the target particle than the starting sample.

Efficiency and throughput of such devices are dependent on the relative intensity of the forces involved. The electric field and field gradient driving the migration process is highly localized and the migration force is small compared to hydrodynamic forces. As a result, the electrode effect region must be sufficiently long for the efficient sorting to take place. As the effect from a single electrode is small compared to the flow effect, arrays of electrodes are positioned along the flow of the channel to maximize the electric field forces, without impacting throughput. In conventional devices, the electrodes are placed at the bottom surface or walls of a fluidic system since it is easy to pattern electrical traces on a flat surface. The flow profile within a typical fluid channel is shown in FIGS. 7A-7B. It is clear that these electrodes are in the region of lowest flow.

### BRIEF SUMMARY OF THE INVENTION

It is an objective of the present invention to provide systems, devices, and methods that allow for sorting particles using electric fields, as specified in the independent claims. Embodiments of the invention are given in the dependent claims. Embodiments of the present invention can be freely combined with each other if they are not mutually exclusive.

This invention describes a device intended to sort small particles, such as cells, utilizing one or more electric fields in a moving fluid. It utilizes a cavity intended to guide the flow of fluid and suspended electrical conductors that are positioned in non-zero flow regions of the flow stream. Ideal use of this invention would be for sorting small particles such as animal or plant cells in a fluid such as water. As particles flow in the fluid, electric fields on the suspended conductors move the small particles from one flow region to another, allowing them to be redirected to different endpoints.

One of the unique and inventive technical features of the present invention is the use of one or more suspended electrical conductors in a flowing stream of fluid for the purpose of separating small particles in a fluid carrier. Furthermore, the electrode conductors are moved from the bottom or edge of the flow channel to the middle of the flow system. Without wishing to limit the invention to any theory or mechanism, it is believed that the technical feature of the present invention improves upon conventional electric field sorting devices by moving the electrodes from the bottom surfaces or walls of a flow channel to the middle of the flow system, utilizing suspended electrodes.

The electrodes at the bottom or edges are particularly troublesome since: (1) cells that are far from the bottom are unaffected; (2) flow velocities are very low at the walls of the flow channel (due to the “no-slip” condition of fluidics), making it difficult for the fluid flow to drag the cells into the collection stream; (3) electrodes on the bottom surface also reduce the electric field coverage, since the field is unused below the channel surface; (4) patterning metal electrodes on surfaces is generally a very expensive manufacturing process. None of the presently known prior references or work has the unique inventive technical feature of the present invention.

The inventive features of the present invention improve all the shortcomings of the current art listed prior. Electrode conductors may be suspended by forming wires under tension, as illustrated in FIG. 1. Structural metal may be used to form suspended structures that do not require the use of tension. Suspended conductors may be designed to interact with conductors that are not suspended, such as a conductive wall of the flow channel, as shown in FIG. 2. Flow channels may be of any geometry and more than one conducting element may be used.

Placement of small diameter, cylindrically symmetric wires near the center of a flow stream (as shown in FIGS. 1 and 2), allows one to produce a large electric field that has a clean, symmetric field shape with well-defined gradients. Equally important, the placement of the wire electrodes in the center of the flow stream positions them in the region of greatest velocity. Since many fluidic sorter designs require the fluid flow to drag cells along the electrodes towards the collection stream, it is advantageous for the conducting electrodes to be positioned in a region of high fluid velocity.

Complex geometries of electrodes may be produced using multiple electrodes. Electrodes may be constructed of structurally rigid materials so that they may be formed into useful shapes and suspended without the need for tension. In addition to the use of multiple electrodes, additional conductors may be attached or coated on nearby surfaces if more field shaping is required. FIG. 3 shows a third embodiment with multiple suspended, free-standing electrodes in the central region of the flow stream.

The current invention can be utilized to create complex electric fields in flow systems of any size. This type of structure can be produced in a microfluidic form factor if desired. This small volume format is particularly useful for performing experiments when developing assays that utilize electric fields. In such a case, the suspended conducting elements may be laminated into a small fluidic system to provide the electric fields. FIG. 4 shows an embodiment where electrodes are suspended within a microfluidic laminate.

A cartridge utilizing this invention can be manufactured readily using industry standard processes. There are many ways to construct such a device. For example, the structural components can be injection molded, wiring can be done using wire assembly techniques, and electronic routing can be done with printed circuit board manufacturing (PCB). The use of PCB processing allows low-cost, standard electrical connectors to be attached (such as micro-USB). Additional electronics can be attached if necessary. Other approaches, such as laminating layers and even conventional assembly are also envisioned.

One of the unique and inventive technical features of the present invention is the placement of an electrical conductor at a center axis of a channel. Without wishing to limit the invention to any theory or mechanism, it is believed that the technical feature of the present invention advantageously provides for the production of a large electric field that has a clean, symmetric field shape with well-defined gradients. Furthermore, the placement of the wire electrodes in the center of the flow stream positions them in the region of greatest velocity. None of the presently known prior references or work has the unique inventive technical feature of the present invention.

Furthermore, the inventive technical feature of the present invention is counterintuitive. The reason that it is counterintuitive is because it contributed to a surprising result. One skilled in the art would not implement an electrical conductor that is not placed on a wall outside of bulk fluid flow in a microfluidic device implementing laminar flow. This is due to the fact that microfluidic devices are usually most efficiently constructed through integrated circuit manufacturing techniques (thick film lithography, etching, etc.). This limits the construction of electrodes to the channel walls. Surprisingly, the present invention is fabricated in such a way that is both efficient, and allows for the placement of electric conductors at a center axis of a microfluidic channel. Thus, the inventive technical feature of the present invention contributed to a surprising result and is counterintuitive.

Any feature or combination of features described herein is included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a perspective view of the simplest embodiment of the current invention. This construction enables electric field-assisted sorting at high rates and high performance. The unit may connect to standard electrical and fluidic interfaces and can handle high volumetric flow rates. The device can be manufactured using materials processes that are readily available, including injection molding, wire-bonding, and integration of printed circuit boards.

FIG. 2 shows a perspective view of a second embodiment of the current invention. This construction has a suspended conducting wire in the center of a flow channel having conductive walls. This embodiment enables electric field-assisted sorting at high rates and high performance.

FIG. 3 shows an illustration of an embodiment with multiple suspended, free-standing electrodes in the central region of a flow stream.

FIG. 4 shows an illustration of an embodiment with suspended electrodes in a laminated microfluidic structure. This type of device is useful for low-throughput applications such as assay development and experimental work.

FIG. 5 shows the basic operation of a typical Laminar flow sorter, often found in microfluidic devices. The sorting system leverages the laminar flow nature of small-sized flow systems to enrich a sample. In this example, Inlet B contains a sample of first and second particles. Inlet A contains a fluid devoid of these particles. The two flow streams (A and B) enter from the inlets and form a laminar flow stream that does not mix. An external influence (such as an applied electric field) selectively causes first particles from the “B” stream to drift into the “A” stream. At the end, the “B” stream is populated with the first particles, but not with the second particles, thus representing an enriched population of cells.

FIG. 6 shows an illustration of basic electric field phenomena on small particles. Particles may have a net charge or may be polarized in the presence of an external electric field (as shown in this illustration by two wires having positive and negative charge). The electric field will create a force on charged particles. If there is a gradient in the electric field, the dipole particles will experience a force in the direction of the gradient.

FIG. 7A shows a typical dielectrophoresis field-flow fractionation device. A heterogeneous sample flows into a device through branch A, mixing with a buffer solution from branch B. Each sample is subjected to forces from the electric field between the electrodes. Particles with attractive forces or predominantly hydrodynamic forces acting on them are moved into branch C, while particles with repulsive forces acting on them are moved into branch D.

FIG. 7B shows a parabolic flow profile within a laminar fluid bath. The forces acting on each particle in the flow include the DEP force from the electric field gradient, gravity, and hydrodynamic forces.

FIG. 8 shows a flow chart of a method for sorting particles implementing the device of the present invention.

### DETAILED DESCRIPTION OF THE INVENTION

Following is a list of elements corresponding to a particular element referred to herein:

100 particle sorting device

110 tube

112 channel

114 suspended electrical conductor

120 conductive wall

200 microfluidic device

202 inlet port

204 outlet port

206 fluidic microchannel

210 top layer

212 first channel

214 suspended electrical conductor

215 channel layer

218 second channel

220 bottom layer

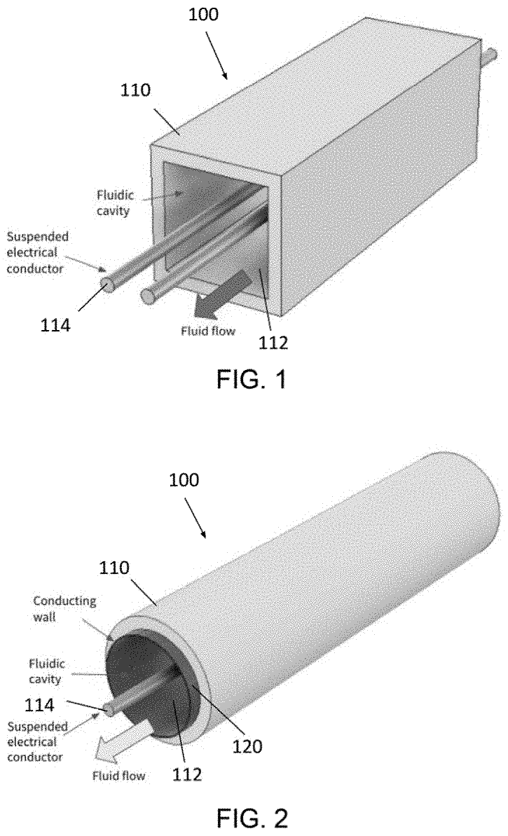

Referring now to FIG. 1, in preferred embodiments, the present invention features a device (100) for sorting particles. The device (100) comprises a tube (110) having a channel (112) therein and at least one electrical conductor (114). The channel (112) is filled with a liquid, and the liquid contains at least two types of particles. The at least one electrical conductor (114) may be longitudinally disposed at a center axis in the channel (112). In some embodiments, the liquid flows through the channel (112). An electrical signal may then be applied to the at least one electrical conductor (114) to generate an electric field, thereby facilitating the sorting of at least one type of particle of the at least two types of particles by pushing at least one type of particle in one direction along the channel (112).

In further embodiments, the at least one electrical conductor (114) is disposed at a center axis of the channel (112). The at least one electrical conductor (114) may be a wire and the wire may be under tension. The device (100) may further comprise a conductive wall (120) disposed throughout an inner surface of the tube (110). In some embodiments, a conductive coating may be disposed throughout an inner surface of the tube (110). In some embodiments, the at least one electrical conductor (114) may be attached to a side of the channel (112). In some embodiments, the device (100) may further comprise a second electrical conductor attached to a side of the channel (112). In other embodiments, the device (100) may further comprise a second electrical conductor disposed at a center axis of the channel (112). Each electrical conductor of the one or more electrical conductors may combine to contribute to a single electrical field. Particle movement is driven by the gradient of the electric field and multiple electrodes/conductors may be used to tailor the electric field gradient. This allows multiple different particle populations to be sorted or particle populations to be sorted using different stimuli leading to a sub-population from the primary one. The electrical field generated may be positively charged or negatively charged. In some embodiments, the liquid may contain a second type of particle such that the second type of particle is pushed in a second direction opposite the direction the first type of particle was pushed. In other embodiments, the second type of particle is pushed in the same direction as the first type of particle such that the first type of particle and the second type of particle travel into separate collection chambers. In other embodiments still, the second type of particle may be held in place within the channel as the first type of particle is pushed along the channel. Each particle may be less than about 100 μm in diameter.

The at least one electrical conductor (114) may be suspended by forming wires under tension, as illustrated in FIG. 1. Structural metal may be used to form suspended structures that do not require the use of tension. Suspended conductors may be designed to interact with conductors that are not suspended, such as a conductive wall (120) of the channel (112), as shown in FIG. 2. The channel (112) may be of any geometry and more than one conducting element may be used. The at least one electrical conductor (114) may comprise copper, gold, platinum, any other conductive material, or a combination thereof.

Placement of small diameter, cylindrically symmetric wires near the center of a flow stream (as shown in FIGS. 1-2), allows one to produce a large electric field that has a clean, symmetric field shape with well-defined gradients. In some embodiments, the electrodes can have a variety of shapes, including rectangular cross-section or any other polygon. Equally important, the placement of the wire electrodes in the center of the flow stream positions them in the region of greatest velocity. Since many fluidic sorter designs require the fluid flow to drag cells along the electrical conductors towards the collection stream, it is advantageous for the electrical conductors to be positioned in a region of high fluid velocity.

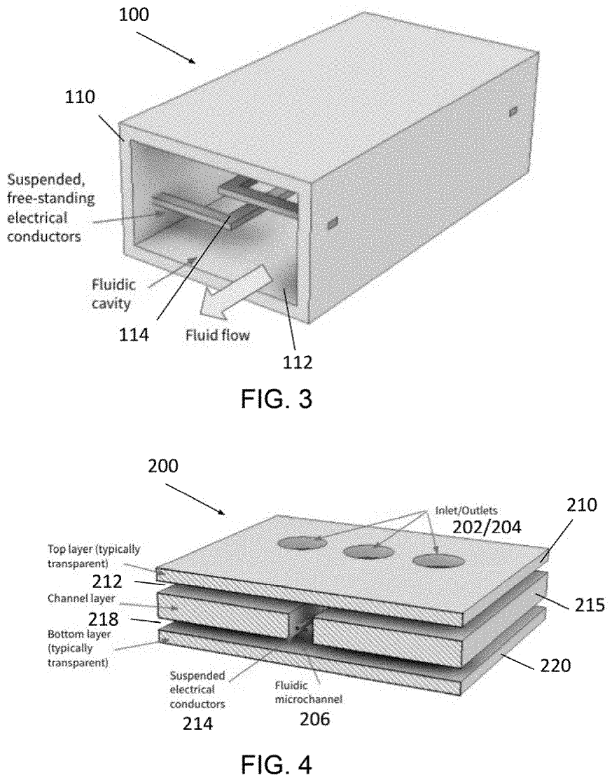

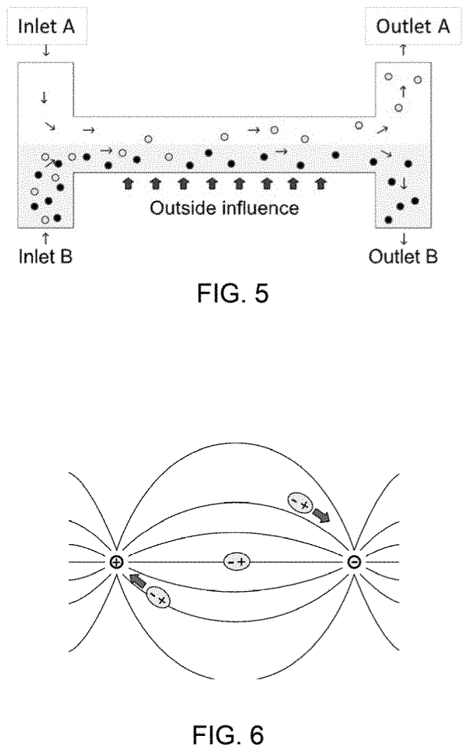

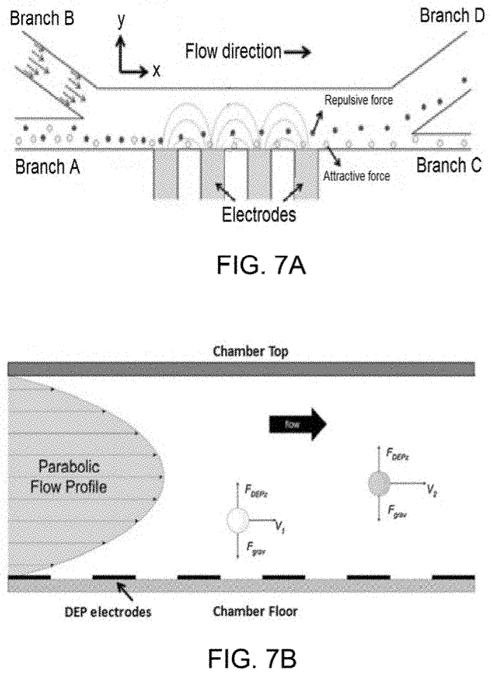

Complex geometries of electrical conductors may be produced using multiple electrical conductors. Electrical conductors may be constructed of structurally rigid materials so that they may be formed into useful shapes and suspended without the need for tension. In addition to the use of multiple electrical conductors, additional conductors may be attached or coated on nearby surfaces if more field shaping is required. Particles are moved in response to the electric field gradient. By shaping the field, the movement and speed of the particles can be better controlled and optimized for the separation and specific particles. FIG. 3 shows a third embodiment with multiple suspended, free-standing electrical conductors in the central region of the flow stream.

The current invention can be utilized to create complex electric fields in flow systems of any size. This type of structure can be produced in a microfluidic form factor if desired. This small volume format is particularly useful for performing experiments when developing assays that utilize electric fields. In such a case, the suspended conducting elements may be laminated into a small fluidic system to provide the electric fields. FIG. 4 shows an embodiment where electrical conductors are suspended within a microfluidic laminate.

A cartridge utilizing this invention can be manufactured readily using industry standard processes. There are many ways to construct such a device. For example, the structural components can be injection molded, wiring can be done using wire assembly techniques, and electronic routing can be done with printed circuit board manufacturing (PCB). The use of PCB processing allows low-cost, standard electrical connectors to be attached (such as micro-USB). Additional electronics can be attached if necessary. Additional electronics may comprise microntrollers/control elements for operating fluid flow and electric conductors, electrical signal generators and sensor, and power electronics for providing energy to other additional electronics. Other approaches, such as laminating layers and even conventional assembly are also envisioned.

In some embodiments, each particle may be less than about 100 μm in diameter. Non-limiting examples of the types of particles include animals cells, plant cells, bacteria, artificially-made particles, fungal cells, biomolecules, naturally derives particles, or a combination thereof. A non-limiting example of a liquid may include water, an enzyme solution, blood, or a combination thereof.

In some embodiments, the present invention features a microfluidic device (200) for sorting particles. The microfluidic device (200) comprises a top layer (210), a channel layer (215), and a bottom layer (220). A first channel (212) is disposed between the top layer (210) and the channel layer (215). A microfluidic channel (206) is disposed in the channel layer (215), and at least one electrical conductor (214) is disposed in the microfluidic channel (206). A second channel (218) is disposed between the channel layer (215) and the bottom layer (220). In other embodiments, the top layer (210) has at least one inlet (202) and at least one outlet (204). In some embodiments, the bottom layer (220) has at least one inlet (202) and at least one outlet (204). In yet another embodiment, the top layer (210) has at least one inlet (202), and the bottom layer (220) has at least one outlet (204). In some embodiments, the device (100) of the present invention may be incorporated into any microfluidic device design. In some embodiments, the inlet (202) of the microfluidic device (200) may be coupled to an outlet of a separate microfluidic device such that particles in the fluid directed through the separate microfluidic device are sorted. In some embodiments, the at least one outlet (204) of the microfluidic device (200) may be coupled to at least one inlet of at least one separate microfluidic device such that particles in the fluid are sorted prior to entering the at least one separate microfluidic device.

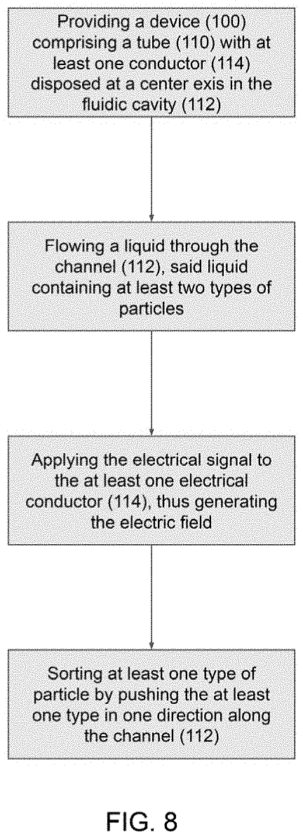

Referring now to FIG. 8, the present invention features a method for sorting particles. The method may comprise providing a device (100). The device (100) may comprise a tube (110) having a channel (112) therein, and at least one electrical conductor (114) longitudinally disposed at a center axis in the channel (112). Applying an electrical signal to the at least one electrical conductor (114) may generate an electric field. The electric field may facilitate the sorting of the at least one type of particle of at least two types of particles by pushing the at least one type of particle in one direction along the channel (112). The method may further comprise flowing a liquid through the channel (112), said liquid containing at least two types of particle, applying the electrical signal to the at least one electrical conductor (114), thus generating the electric field, and sorting at least one type of particle of the at least two types of particles by pushing the at least one type in one direction along the channel (112). In some embodiments, the at least two types of particles may comprise animal cells, plant cells, or a combination thereof. The liquid may be water. The at least one electrical conductor (114) may be a wire and the wire may be under tension. The device (100) may further comprise a conductive wall (120) disposed throughout an inner surface of the tube (110). The device (100) may further comprise a conductive coating disposed throughout an inner surface of the tube (110). The device (100) may further comprise a second electrical conductor attached to a side of the channel (112). Each particle may be less than about 100 μm in diameter.

As used herein, the term “about” refers to plus or minus 10% of the referenced number. Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A device (100) for sorting particles, the device comprising:
a. a tube (110) having a channel (112) therein, wherein the channel (112) is filled with a liquid, said liquid containing at least two types of particles; and
b. at least one electrical conductor (114) longitudinally disposed at a center axis in the channel (112);
wherein the liquid flows through the channel (112);
wherein an electrical signal is applied to the at least one electrical conductor (114) to generate an electric field;
wherein the electric field facilitates the sorting of at least one type of particle of the at least two types of particles by pushing the at least one type of particle in one direction along the channel (112).

2. The device (100) of claim 1, wherein the at least two types of particles comprise animal cells, plant cells, or a combination thereof.

3. The device (100) of claim 1, wherein the liquid is water.

4. The device (100) of claim 1, wherein the at least one electrical conductor (114) is a wire.

5. The device (100) of claim 5, wherein the wire is under tension.

6. The device (100) of claim 1, wherein the device (100) further comprises a conductive wall (120) disposed throughout an inner surface of the tube (110).

7. The device (100) of claim 1, wherein the device (100) further comprises a conductive coating disposed throughout an inner surface of the tube (110).

8. The device (100) of claim 1 further comprising a second electrical conductor attached to a side of the channel (112).

9. The device (100) of claim 1, wherein each particle of the at least two types of particles is less than about 100 μm in diameter.

10. A microfluidic device (200), the microfluidic device comprising:
a. a top layer (210);
b. a channel layer (215) disposed below the top layer (210), wherein a first channel (212) is disposed between the top layer (210) and the channel layer (215);
c. a microfluidic channel (206) disposed in the channel layer (215), wherein at least one electrical conductor (214) is disposed at a center axis in the microfluidic channel (206); and
d. a bottom layer (220) disposed below the channel layer (215), wherein a second channel (218) is disposed between the channel layer (215) and the bottom layer (220).

11. The microfluidic device (200) of claim 10, wherein the top layer (210) has at least one inlet (202), and the bottom layer (220) has at least one outlet (204).

12. A method for sorting particles, the method comprising:
a. providing a device (100) comprising:
i. a tube (110) having a channel (112) therein, wherein the channel (112) configured to be filled with a liquid, said liquid containing at least two types of particles; and
ii. at least one electrical conductor (114) longitudinally disposed at a center axis in the channel (112);
wherein applying an electrical signal to the at least one electrical conductor (114) generates an electric field;
wherein the electric field facilitates the sorting of at least one type of particle of the at least two types of particles by pushing the at least one type of particle in one direction along the channel (112);
b. applying the electrical signal to the at least one electrical conductor (114), thus generating the electric field; and
c. flowing the liquid through the channel (112), wherein when the at least one type of particle of the at least two types of particle flows through the electric field, the electric field pushes the at least one type of particle in one direction along the channel, thereby sorting the at least one type of particle.

13. The method of claim 1, wherein the at least two types of particles comprise animal cells, plant cells, or a combination thereof.

14. The method of claim 1, wherein the liquid is water.

15. The method of claim 1, wherein the at least one electrical conductor (114) is a wire.

16. The method of claim 5, wherein the wire is under tension.

17. The method of claim 1, wherein the device (100) further comprises a conductive wall (120) disposed throughout an inner surface of the tube (110).

18. The method of claim 1, wherein the device (100) further comprises a conductive coating disposed throughout an inner surface of the tube (110).

19. The method of claim 1, wherein the device (100) further comprises a second electrical conductor attached to a side of the channel (112).

20. The method of claim 1, wherein each particle of the at least two types of particles is less than about 100 μm in diameter.
